(19) United States
(12) Patent Application Publication
(10) Pub. No.: US 2012/0271252 A1
(43) Pub. Date: Oct. 25, 2012

(54) MEDICAL FLUID NOZZLE

(75) Inventor: Tadashi Otsuka, Hyogo (JP)

(73) Assignee: Senju Pharmaceutical Co., Ltd., Osaka (JP)

(21) Appl. No.: 13/497,313

(22) PCT Filed: Sep. 24, 2010
(86) PCT No.: PCT/JP2010/066523

§ 371 Date: Mar. 21, 2012

(30) Foreign Application Priority Data
Sep. 24, 2009 (JP) 2009-219155

(51) Int. Cl. A61F 9/00; A61M 35/00

(52) U.S. Cl. 604/294

(57) ABSTRACT

Disclosed is a medical fluid nozzle which makes fluid drops less likely to remain at its tip portion after the medical fluid is dropped. The medical fluid nozzle 1 is a substantially cylindrical shape with a tip surface formed in a substantially planar shape. An annular ridge 10 is formed along the circumferential direction at the tip portion of the medical fluid nozzle 1. The ridge 10 is formed so as to be continuous with the tip surface of the medical fluid nozzle 1 and has an extended surface 11 formed with an increased diameter so as to be flush with the tip surface of the medical fluid nozzle 1.

### BACKGROUND OF THE INVENTION

1. Field of the Invention

The present invention relates to a medical fluid nozzle for causing a medical fluid in a container to fall in drops.

2. Description of the Related Art

Conventionally, to a spout B of a medical fluid container A in which a medical fluid such as eye drops is housed, a nozzle C to cause the medical fluid to fall in drops is attached, as shown in FIG. 8. The nozzle C is made mainly from low-density polyethylene, formed at its tip portion in a substantially hemispherical shape, and formed flat at a tip of the hemispherical portion.

When dropping the medical fluid using the medical fluid container A with the nozzle C attached, in a state where the medical fluid container A is held horizontally or at a nearly horizontal angle, the medical fluid may remain in the nozzle C without being dropped. In a state where the medical fluid container A is held vertically or at a nearly vertical angle, fluid drops may slightly remain at a tip part of the nozzle C even when the medical fluid has been dropped.

The following patent document 1 discloses a nozzle to be inserted in a cylindrical spout neck portion of a container main body, an upper side of an upper portion of the nozzle is formed in a hemispherical shape, and a ring-shaped protrusion is formed at an outer peripheral surface of a maximum outer diameter part of the hemispherical portion. Since the ring-shaped protrusion is formed at the maximum outer diameter part of the hemispherical portion of the nozzle, the fluid stops satisfactorily. Fluid drops are easily formed in not only a state where the nozzle is turned almost directly downward but also a state where the nozzle is turned directly sideways or inclined obliquely downward, so that the medical fluid can be caused to fall in drops via the ring-shaped protrusion of the nozzle.

It is demanded of a nozzle of a medical fluid container from the viewpoint of preventing contamination that no fluid drops remain in the nozzle after the medical fluid is dropped, dripping can be prevented even if fluid drops remain, and the occurrence of a flash is suppressed, and from the viewpoint of production efficiency, not to use a split mold for manufacturing the nozzle.
Patent Document 1: Japanese Patent No. 3971329

### SUMMARY OF THE INVENTION

It is an object of the present invention to provide a medical fluid nozzle which makes fluid drops less likely to remain at its tip portion after the medical fluid is dropped and which can be molded without using a split mold.

The present invention has been made in order to achieve the object described above, and a first aspect of the present invention is a medical fluid nozzle which is a cylindrical medical fluid nozzle to be attached to a spout of a medical fluid container inside of which a medical fluid is housed, for causing the medical fluid to fall in drops, and the medical fluid nozzle is formed in a substantially planar shape at a tip surface thereof, and is formed at a tip portion thereof with an annular ridge along a circumferential direction, and the ridge is formed so as to be continuous with the tip surface, and has an extended surface formed with an increased diameter so as to be flush with the tip surface or formed increased in diameter toward a base end side.

A second aspect of the present invention is the medical fluid nozzle according to the first aspect of the present invention, in which the ridge has an external shape that is increased in diameter so as to be flush with the tip surface, extends to a base end side, and is then reduced in diameter toward the base end side.

A third aspect of the present invention is the medical fluid nozzle according to the first aspect of the present invention, in which the ridge has an external shape that is increased in diameter so as to be flush with the tip surface and is then reduced in diameter toward a base end side.

A fourth aspect of the present invention is the medical fluid nozzle according to the first aspect of the present invention, in which the ridge has an external shape that is increased in diameter toward a base end side and is then reduced in diameter toward the base end side.

A fifth aspect of the present invention is an eye drop container including the medical fluid nozzle according to any one of the first to fourth aspects of the present invention.

Further, a sixth aspect of the present invention is a method for preventing dripping from a medical fluid nozzle for which the medical fluid nozzle according to any one of the first to fourth aspects of the present invention is attached to a spout of a medical fluid container.

According to the present invention, a medical fluid nozzle which makes fluid drops less likely to remain at its tip portion after the medical fluid is dropped and which can be molded without using a split mold can be provided.

### BRIEF DESCRIPTION OF THE DRAWINGS

FIG. 1 is a schematic front view showing Embodiment 1 of a medical fluid nozzle of the present invention, showing a part thereof in section,

FIG. 2 is a schematic front view showing a usage state of the medical fluid nozzle of FIG. 1,

FIG. 3 is a view showing Embodiment 2 of a medical fluid nozzle of the present invention attached to a spout of a medical fluid container, showing a part thereof in section,

FIG. 4 is a schematic front view showing Embodiment 3 of a medical fluid nozzle of the present invention, showing a part thereof in section,

FIG. 5 is a schematic front view showing Embodiment 4 of a medical fluid nozzle of the present invention, showing a part thereof in section,

FIG. 6 is a schematic view showing a laboratory instrument to clarify the effect of a medical fluid nozzle of the present invention,

FIG. 7 is a front view showing a nozzle of a comparative example to clarify the effect of a medical fluid nozzle of the present invention, showing a part thereof in section,

FIG. 8 is a view showing a conventional medical fluid nozzle attached to a spout of a medical fluid container, showing a part thereof in section.

### DESCRIPTION OF THE PREFERRED EMBODIMENTS

Hereinafter, embodiments of the present invention will be described in detail with reference to the drawings.

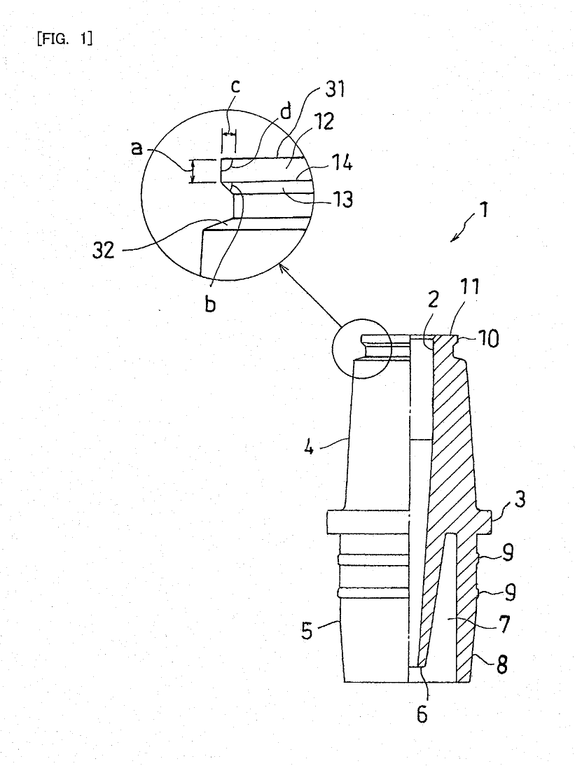

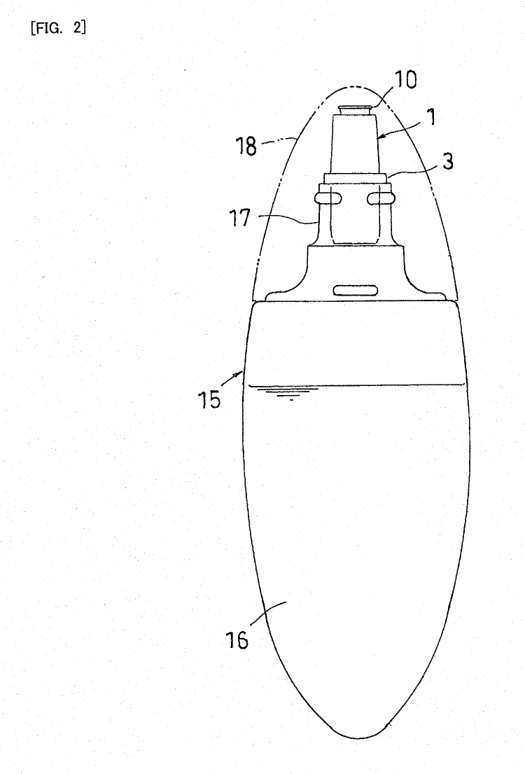

FIG. 1 and FIG. 2 are schematic views showing Embodiment 1 of a medical fluid nozzle of the present invention, in which FIG. 1 is a front view showing a part thereof in section, and FIG. 2 is a front view showing a usage state thereof. The medical fluid nozzle is attached to a medical fluid container and causes a medical fluid housed inside the medical fluid container to fall in drops.

The medical fluid nozzle 1 is made from low-density polyethylene, and is integrally formed by injection molding. The medical fluid nozzle 1 is formed in a substantially circular cylindrical shape that is opened at the top and bottom. Specifically, an outer peripheral surface of the medical fluid nozzle 1 is formed in a shape that is gradually increased in diameter toward its tip side up to a substantially middle portion in its axial direction and is then reduced in diameter toward the tip side.

An inner hole 2 of the medical fluid nozzle 1 is formed in a columnar shape at its tip portion, while a base end portion further than the tip portion is formed in a shape that is reduced in diameter toward the base end side. Of the inner hole 2 of the medical fluid nozzle 1, the tip portion of the inner hole formed in a columnar shape is formed in a substantially inverse circular truncated cone shape that is increased in diameter toward the tip side.

The medical fluid nozzle 1 is integrally formed at a substantially middle portion in its axial direction with a plate piece 3 that is extended in a flange shape from the outer peripheral surface. The medical fluid nozzle 1 has an upper cylinder portion 4 disposed on the tip side of the plate piece 3, an upper end cylinder portion 31 disposed on the tip side of the upper cylinder portion 4, and a lower cylinder portion 5 disposed on the base end side of the plate piece 3. The lower cylinder portion 5 includes a substantially circular cylindrical inner cylinder portion 6 and a substantially circular cylindrical outer cylinder portion 8 disposed so as to form a circular cylindrical gap 7 with the inner cylinder portion 6.

An outer peripheral surface of the inner cylinder portion 6 is formed in a shape that is reduced in diameter toward the base end side. The inner hole of the inner cylinder portion 6 forms a base end portion side of the inner hole 2 of the medical fluid nozzle 1. At an outer peripheral surface of the outer cylinder portion 8, an annular projection 9 is formed along the circumferential direction. In the illustrated example, the projection 9 is formed to show a substantially rectangular shape in section, and two projections 9, 9 are formed isolated from each other in the axial direction on the outer peripheral surface of the outer cylinder portion 8. A base end surface of the outer cylinder portion 8 is located on the base end side further than a base end surface of the inner cylinder portion 6.

The upper end cylinder portion 31 of the medical fluid nozzle 1 is formed from a tip portion of the upper cylinder portion 4 to the tip side via a circular truncated cone shaped portion 32. Specifically, an outer peripheral surface of the tip of the medical fluid nozzle 1 has a shape that extends to the tip side after being reduced in diameter from the tip portion of the upper cylinder portion 4 toward the tip side, and then extends to the tip side after being increased in diameter toward the tip side. That is, at a tip portion of the medical fluid nozzle 1, an annular ridge 10 showing a substantially rectangular shape in section is formed along the circumferential direction. Specifically, the ridge 10 has an external shape that is increased in diameter so as to be flush with a tip surface of the medical fluid nozzle 1 formed in a substantially planar shape, extends to the base end side, and is then reduced in diameter toward the base end side. In the illustrated example, a maximum outer diameter portion of the ridge 10 is formed so as to have an outer diameter smaller than that of the upper cylinder portion 4 of the medical fluid nozzle 1.

Thus, the ridge 10 of the medical fluid nozzle 1 is formed so as to be continuous with the tip surface of the medical fluid nozzle 1, and has an extended surface 11 formed with an increased diameter so as to be flush with the tip surface of the medical fluid nozzle 1. The extended surface 11 and the tip surface of the medical fluid nozzle 1 form, in the medical fluid nozzle 1, a drop forming surface to form fluid drops to be dropped.

The ridge 10 of the medical fluid nozzle 1 has a thickness “a” of approximately 0.3 mm at its predetermined portion. The ridge 10 of the medical fluid nozzle 1 has a columnar shape at its tip portion, and the height of this columnar shaped portion 12 is provided as the predetermined portion thickness “a” of the ridge 10. The ridge 10 of the medical fluid nozzle 1 has an angle “b” of approximately 33.5° at its predetermined portion. The ridge 10 of the medical fluid nozzle 1 has an inverse circular truncated cone shape at its base end portion, and an angle created by an outer peripheral surface of this inverse circular truncated cone shaped portion 13 and a tip surface 14 of the inverse circular truncated cone shaped portion 13 is provided as the predetermined portion angle “b” of the ridge 10.

As shown in FIG. 2, the medical fluid container 15 is a container inside of which a medical fluid 16 is housed. The medical fluid container 15 is a thin-walled hollow container that can be deformed by pressing, and is integrally formed of plastic. The medical fluid container 15 has a circular cylindrical spout 17 at its tip portion. The spout 17 is formed to have an inner diameter slightly smaller than the outer diameter of the projection 9 of the medical fluid nozzle 1.

To the spout 17 of the medical fluid container 15, the medical fluid nozzle 1 is attached. Specifically, the medical fluid nozzle 1 is attached by fitting the outer cylinder portion 8 of the medical fluid nozzle 1 into the spout 17 until the plate piece 3 of the medical fluid nozzle 1 abuts against a tip surface of the spout 17 of the medical fluid container 15. As mentioned above, the projection 9 of the medical fluid nozzle 1 is formed to have an outer diameter slightly larger than the inner diameter of the spout 17 of the medical fluid container 15. Therefore, the outer cylinder portion 8 of the medical fluid nozzle 1 is forcibly fitted into the spout 17 of the medical fluid container 15. Accordingly, the medical fluid nozzle 1 is prevented from unexpectedly coming out of the spout 17 of the medical fluid container 15. The medical fluid 16 housed inside the medical fluid container 15 is also prevented from leaking between the inner surface of the spout 17 of the medical fluid container 15 and the outer surface of the outer cylinder portion 8 of the medical fluid nozzle 1. On an upper portion of the medical fluid container 15, a cap 18 is removably provided in a manner to cover the medical fluid nozzle 1.

The medical fluid 16 to be housed inside the medical fluid container 15 is, for example, eye drops. In this case, a container constructed as a result of the medical fluid nozzle 1 being attached to the medical fluid container 15 is an eye drop container.

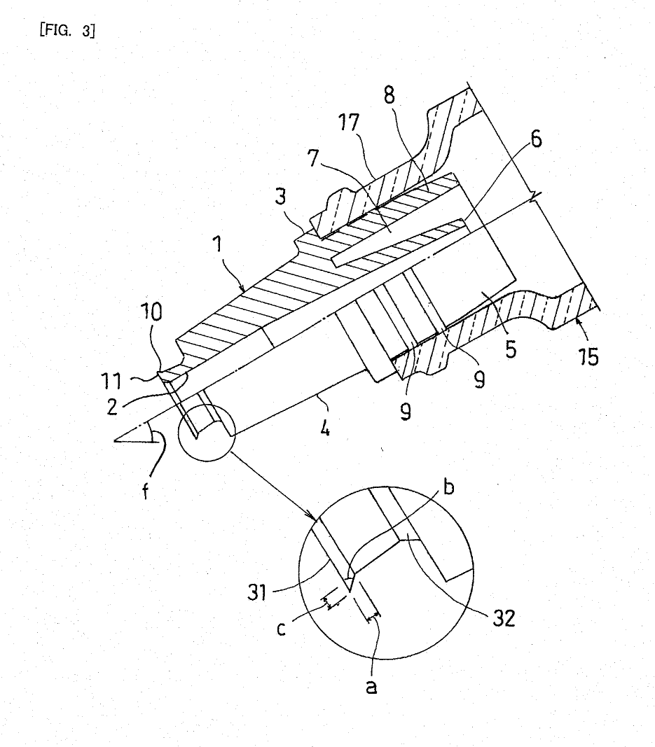

FIG. 3 is a view showing Embodiment 2 of a medical fluid nozzle of the present invention attached to a spout of a medical fluid container, showing a part thereof in section. The medical fluid nozzle 1 of Embodiment 2 has basically the same configuration as that of the medical fluid nozzle of Embodiment 1 described above. Therefore, in the following, description will be given mainly of the differences between the embodiments, and corresponding parts are denoted by the same reference signs.

In Embodiment 2, an outer peripheral surface of the tip of the medical fluid nozzle 1 has a shape that extends to the tip side after being reduced in diameter from a tip portion of the upper cylinder portion 4 toward the tip side, and then is increased in diameter toward the tip side. That is, at a tip portion of the medical fluid nozzle 1, an annular ridge 10 showing a substantially triangular shape in section is formed along the circumferential direction. Specifically, the ridge 10 has an external shape that is increased in diameter so as to be flush with a tip surface of the medical fluid nozzle 1 and is then reduced in diameter toward the base end side. In the illustrated example, a maximum outer diameter portion of the ridge 10 is formed so as to have an outer diameter smaller than that of the upper cylinder portion 4 of the medical fluid nozzle 1.

Thus, the ridge 10 of the medical fluid nozzle 1 is formed so as to be continuous with the tip surface of the medical fluid nozzle 1, and has an extended surface 11 formed with an increased diameter so as to be flush with the tip surface of the medical fluid nozzle 1. The extended surface 11 and the tip surface of the medical fluid nozzle 1 form, in the medical fluid nozzle 1, a drop forming surface to form fluid drops to be dropped.

The ridge 10 of the medical fluid nozzle 1 has a thickness “a” of approximately 0.1 mm at its predetermined portion. In the present embodiment, the predetermined portion thickness “a” of the ridge 10 indicates the thickness of the ridge 10. The ridge 10 of the medical fluid nozzle 1 has an angle “b” of approximately 68.0° at its predetermined portion. In the present embodiment, the predetermined portion angle “b” of the ridge 10 indicates an angle created by an outer peripheral surface of the ridge 10 and the extended surface 11 of the ridge 10.

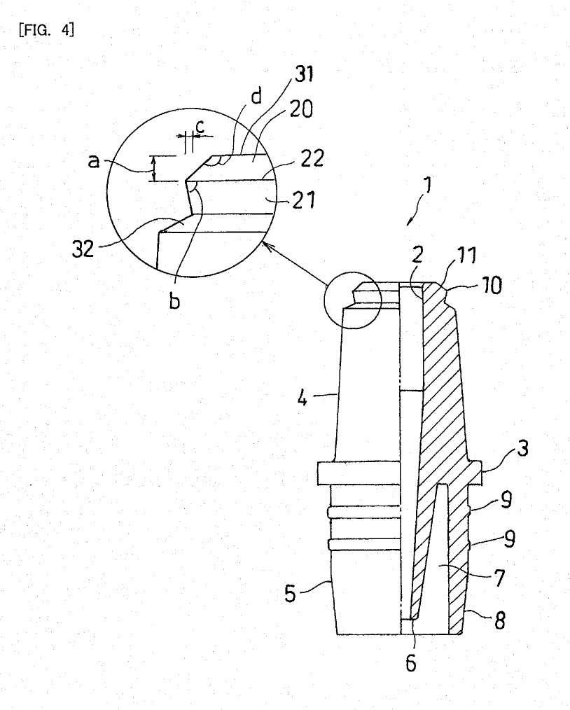

FIG. 4 is a schematic front view showing Embodiment 3 of a medical fluid nozzle of the present invention, showing a part thereof in section. The medical fluid nozzle 1 of Embodiment 3 has basically the same configuration as that of the medical fluid nozzle of Embodiment 1 described above. Therefore, in the following, description will be given mainly of the differences between the embodiments, and corresponding parts are denoted by the same reference signs.

In Embodiment 3, an outer peripheral surface of the tip of the medical fluid nozzle 1 has a shape that is reduced in diameter from a tip portion of the upper cylinder portion 4 toward the tip side, is increased in diameter toward the tip side, and is then reduced in diameter toward the tip side. That is, at a tip portion of the medical fluid nozzle 1, an annular ridge 10 showing a substantially triangular shape in section is formed along the circumferential direction. Specifically, the ridge 10 has an external shape that is increased in diameter toward the base end side and is then reduced in diameter toward the base end side. In the illustrated example, a maximum outer diameter portion of the ridge 10 is formed so as to have an outer diameter smaller than that of the outer peripheral surface of the upper cylinder portion 4 of the medical fluid nozzle 1.

Thus, the ridge 10 of the medical fluid nozzle 1 is formed so as to be continuous with the tip surface of the medical fluid nozzle 1, and has an extended surface 11 formed increased in diameter toward the base end side. In Embodiment 1 described above, the tip surface of the medical fluid nozzle 1 and the extended surface 11 form a drop forming surface, while in the present embodiment, the tip surface of the medical fluid nozzle 1 serves as a drop forming surface.

The ridge 10 of the medical fluid nozzle 1 has a thickness “a” of approximately 0.3 mm at its predetermined portion. The ridge 10 of the medical fluid nozzle 1 has a circular truncated cone shape at its tip portion, and the height of this circular truncated cone shaped portion 20 is provided as the predetermined portion thickness “a” of the ridge 10. The ridge 10 of the medical fluid nozzle 1 has an angle “b” of approximately 69.6° at its predetermined portion. The ridge 10 of the medical fluid nozzle 1 has an inverse circular truncated cone shape at its base end portion, and an angle created by an outer peripheral surface of this inverse circular truncated cone shaped portion 21 and a tip surface 22 of the inverse circular truncated cone shaped portion 21 is provided as the predetermined portion angle “b” of the ridge 10.

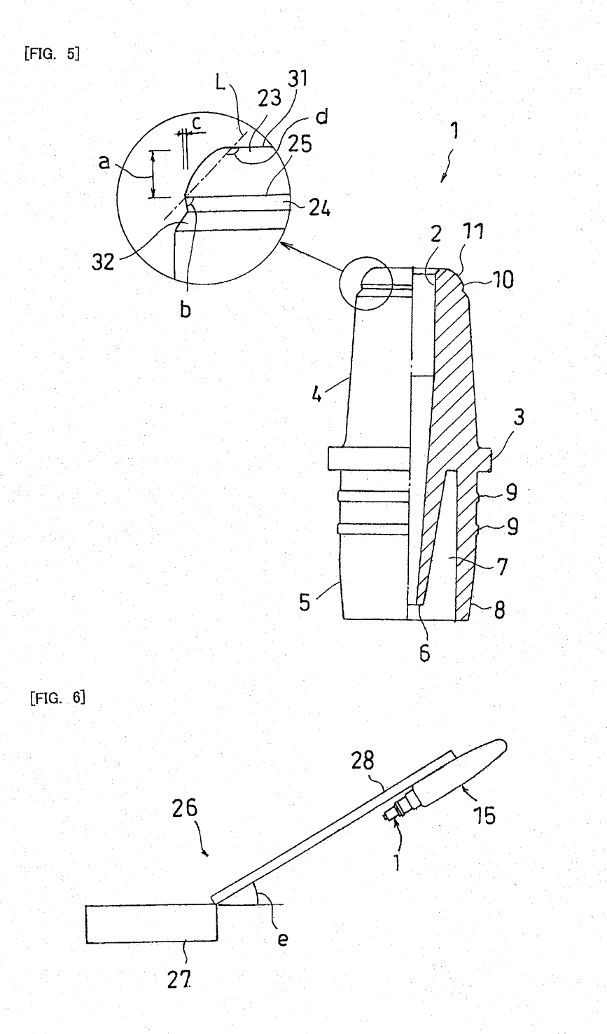

FIG. 5 is a schematic front view showing Embodiment 4 of a medical fluid nozzle of the present invention, showing a part thereof in section. The medical fluid nozzle 1 of Embodiment 4 has basically the same configuration as that of the medical fluid nozzle of Embodiment 1 described above. Therefore, in the following, description will be given mainly of the differences between the embodiments, and corresponding parts are denoted by the same reference signs.

In Embodiment 4, an outer peripheral surface of the tip of the medical fluid nozzle 1 has a shape that is reduced in diameter from a tip portion of the upper cylinder portion 4 toward the tip side, is increased in diameter toward the tip side, and is then gradually reduced in diameter toward the tip side. That is, at a tip portion of the medical fluid nozzle 1, an annular ridge 10 showing a substantially triangular shape in section is formed along the circumferential direction. Specifically, the ridge 10 has an external shape that is gradually increased in diameter toward the base end side and is then reduced in diameter toward the base end side. In the illustrated example, a maximum outer diameter portion of the ridge 10 is formed so as to have an outer diameter slightly smaller than that of the outer peripheral surface of the upper cylinder portion 4 of the medical fluid nozzle 1.

Thus, the ridge 10 of the medical fluid nozzle 1 is formed so as to be continuous with the tip surface of the medical fluid nozzle 1, and has an extended surface 11 formed gradually increased in diameter toward the base end side. In Embodiment 1 described above, the tip surface of the medical fluid nozzle 1 and the extended surface 11 form a drop forming surface, while in the present embodiment, the tip surface of the medical fluid nozzle 1 serves as a drop forming surface.

The ridge 10 of the medical fluid nozzle 1 has a thickness “a” of approximately 0.7 mm at its predetermined portion. The ridge 10 of the medical fluid nozzle 1 has a substantially circular truncated cone shape at its tip portion, and the height of this substantially circular truncated cone shaped portion 23 is provided as the predetermined portion thickness “a” of the ridge 10. And the ridge 10 of the medical fluid nozzle 1 has an angle “b” of approximately 82.1° at its predetermined portion. The ridge 10 of the medical fluid nozzle 1 has an inverse circular truncated cone shape at its base end portion, and an angle created by an outer peripheral surface of this inverse circular truncated cone shaped portion 24 and a tip surface 25 of the inverse circular truncated cone shaped portion 24 is provided as the predetermined portion angle “b” of the ridge 10.

According to the embodiments described above, the annular ridge 10 is formed at the tip portion of the medical fluid nozzle 1. Accordingly, fluid drops are less likely to remain at the tip portion of the medical fluid nozzle 1 when the medical fluid 16 is dropped in a state where the medical fluid container 15 with the medical fluid nozzle 1 attached is held not only vertically or at a nearly vertical angle but also horizontally or at a nearly horizontal angle. When fluid drops remain, even if the medical fluid container 15 in a dropping state is turned into an upright state, dripping of the remaining fluid drops is prevented. Particularly, when a fluid with a low surface tension that easily causes dripping is filled, the effect of preventing dripping by the present embodiment is remarkably exhibited. The number of cases that the medical fluid 16 remains in the medical fluid nozzle without being dropped is significantly reduced when the medical fluid 16 is intended to be dropped in a state where the medical fluid container 15 with the nozzle 1 attached is held horizontally or at a nearly horizontal angle.

Moreover, the tip portion of the medical fluid nozzle 1 does not have a pointed shape. Accordingly, the medical fluid container 15 with the medical fluid nozzle 1 attached is used suitably for applying eye drops. Since each of the medical fluid nozzles 1 of the embodiments described above has the shape mentioned above, it is not necessary to split a cavity mold because a molded item can be released from the mold by extraction in the vertical direction. This prevents the formation of a flash on the outer periphery of the medical fluid nozzle 1. Accordingly, the medical fluid nozzle 1 is attached to the spout 17 of the medical fluid container 15 in a watertight state. Further, each of the medical fluid nozzles 1 of the embodiments described above can be prepared at a low cost since it is not necessary to split the cavity mold.

The medical fluid nozzle of the present invention is not limited to the configurations of the embodiments described above, and can be appropriately modified. For example, in the embodiments described above, the medical fluid nozzle 1 is made from low-density polyethylene, but the material is not particularly limited as long as it is a raw resin that is normally used for injection molding, and for example, linear low-density polyethylene, polypropylene, or the like is used.

The predetermined portion thickness “a” of the ridge 10 of the medical fluid nozzle 1 is normally approximately 0.1 mm to approximately 1.0 mm, and preferably, approximately 0.2 mm to approximately 0.7 mm, and more preferably, approximately 0.3 mm to approximately 0.7 mm. The predetermined portion angle “b” of the ridge 10 of the medical fluid nozzle 1 is normally approximately 5° to approximately 85°, and preferably, approximately 10° to approximately 70°, and more preferably, approximately 20° to approximately 45°. Of the inverse circular truncated cone shaped portion formed at the base end portion of the ridge 10 of the medical fluid nozzle 1 (ridge 10 in Embodiment 2), an outer diameter difference “c” between both axial end surfaces is normally approximately 0.01 mm to approximately 0.2 mm, and preferably, approximately 0.1 mm to approximately 0.2 mm. And an angle “d” created by the drop forming surface formed in the medical fluid nozzle 1 and the outer peripheral surface of the ridge 10 formed in the medical fluid nozzle 1 is normally approximately 80° to approximately 170°, and preferably approximately 90° to approximately 140°. In Embodiment 4, the angle “d” is provided as an angle created by the tip surface of the medical fluid nozzle 1 and a dashed line L being a straight line that connects an outer edge portion of the tip surface of the medical fluid nozzle 1 and the maximum outer diameter portion of the ridge 10.

The surface tension of the medical fluid 16 to be housed in the medical fluid container 15 is normally approximately 30 mN/m to approximately 60 mN/m, and preferably, approximately 35 mN/m to approximately 45 mN/m. Further, the medical fluid 16 to be housed in the medical fluid container 15 is not limited to eye drops, and may be ear drops or nasal drops.

### EXAMPLES

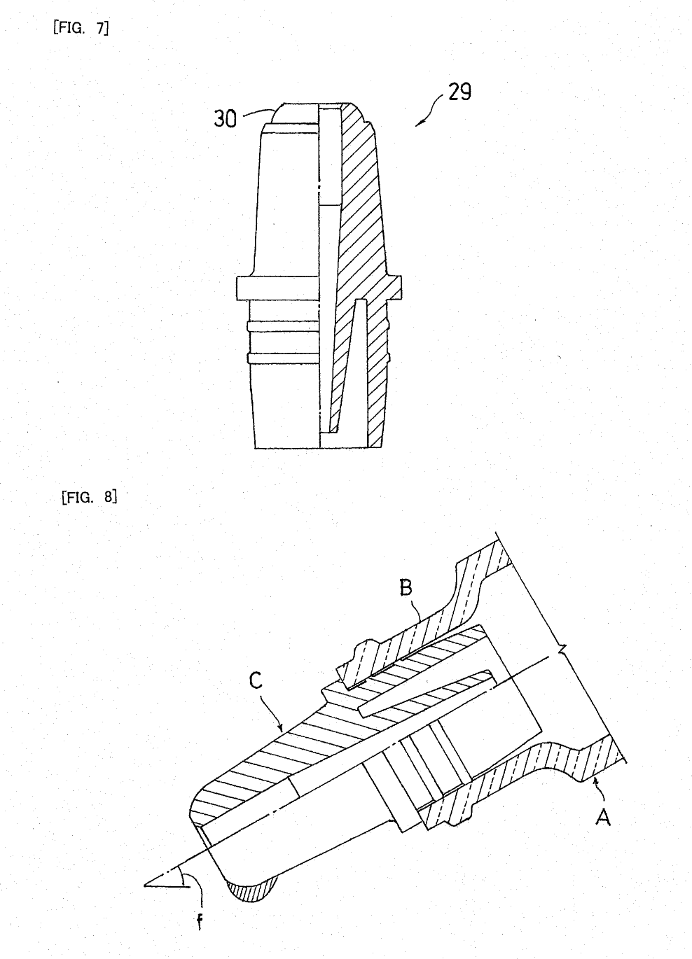

Next, experimentation to clarify the effect of the medical fluid nozzles 1 of the embodiments described above will be described. Table 1 shows measurements of respective portions of the medical fluid nozzles used for the experimentation. FIG. 6 is a schematic view showing a laboratory instrument to clarify the effect of a medical fluid nozzle of the present invention. FIG. 7 is a front view showing a nozzle of Comparative example 2 to clarify the effect of a medical fluid nozzle of the present invention, showing a part thereof in section.

As shown in FIG. 6, the laboratory instrument 26 includes a fixed base 27 disposed horizontally and an arm 28 rotatably provided on the fixed base 27. The arm 28 attached to the fixed base 27 can maintain an angle “e” created with a horizontal plane at a predetermined angle.

As shown in FIG. 7, the nozzle 29 of Comparative example 2 is reduced in diameter at its tip portion to form a small diameter portion 30. The small diameter portion 30 is formed in a substantially hemispherical shape, and formed flat at its tip surface. Other aspects of the configuration are the same as those of the embodiments described above.

When performing the experimentation, first, eye drops were filled in the medical fluid container 15. The eye drops were pH7 and had a surface tension of 42 mN/m (surface tensiometer K100C manufactured by Kruss Company). Then, the medical fluid nozzle 1 was attached to the spout 17 of the medical fluid container 15. Here, description will be given for the case of the medical fluid nozzle 1 of Embodiment 2 described above.

The medical fluid container 15 containing eye drops with the medical fluid nozzle 1 attached was fixed to a tip portion of the arm 28 so that the medical fluid nozzle 1 faces downward when the arm 28 of the laboratory instrument 26 is inclined. At this time, an angle (dropping angle) “f” created by the axis of the medical fluid nozzle 1 and a horizontal plane was the same as the angle “e” created by the arm 28 and a horizontal plane.

Then, one drop was caused to fall at a position where the angle “e” created by the arm 28 of the laboratory instrument 26 and the horizontal plane reached 90°. After the weight of the medical fluid container with the nozzle immediately after dropping was measured, fluid drops adhered to the tip portion of the nozzle were completely wiped away, and the weight of the medical fluid container with the nozzle was again measured to determine the “remaining amount of fluid drops at the tip” by a calculation formula of “the weight of the medical fluid container with the nozzle immediately after dropping minus the weight of the medical fluid container with the nozzle after fluid drops were completely wiped away.” The “remaining amount of fluid drops at the tip” was determined in the same manner also for the cases where the angle “e” created by the arm 28 of the laboratory instrument 26 and the horizontal plane is 60°, 30°, and 0°, respectively.

Experimentation was performed in the same manner also for the medical fluid nozzles 1 of Embodiments 1, 3, and 4, the nozzle C of Comparative example 1 of FIG. 8, and the nozzle 29 of Comparative example 2 of FIG. 7. Table 2, which is a table showing experimental results, shows the remaining amounts (unit: mg) of fluid drops at the tips of the nozzles after one drop was caused to fall. Each remaining amount of fluid drops is an average value of ten examples for each of the dropping angles “f,” and is expressed by “average value±standard deviation.”

TABLE 1

| | | | Angle d (°) | |
|---|---|---|---|---|
| | | | created by | |
| | Pre- | | drop forming | |
| | determined | Pre- | surface and | |
| | portion | determined | outer | Outer |
| | thickness | portion | peripheral | diameter |
| | a (mm) | angle b | surface | difference c |
| | of ridge | (°) of ridge | of ridge | (mm) |
| Embodiment 1 | 0.3 | 33.5 | 96.2 | 0.15 |
| Embodiment 2 | 0.1 | 68.0 | — | 0.09 |
| Embodiment 3 | 0.3 | 69.6 | 144.2 | 0.13 |
| Embodiment 4 | 0.7 | 82.1 | 136.0 | 0.02 |

TABLE 2

| | 0° | 30° | 60° | 90° |
|---|---|---|---|---|
| Embodiment 1 | 0.0 ± 0.0 | 0.0 ± 0.0 | 0.0 ± 0.0 | 0.0 ± 0.0 |
| Embodiment 2 | 6.9 ± 1.2 | 0.0 ± 0.0 | 0.0 ± 0.0 | 0.0 ± 0.0 |
| Embodiment 3 | 5.0 ± 1.2 | 0.0 ± 0.0 | 0.0 ± 0.0 | 0.0 ± 0.0 |
| Embodiment 4 | 5.6 ± 1.0 | 0.0 ± 0.0 | 0.0 ± 0.0 | 0.0 ± 0.0 |
| Comparative | 8.9 ± 0.4 | 0.7 ± 0.1 | 0.0 ± 0.0 | 0.0 ± 0.0 |
| example 1 | | | | |
| Comparative | 5.6 ± 0.8 | 1.5 ± 0.4 | 0.0 ± 0.0 | 0.0 ± 0.0 |
| example 2 | | | | |

As can be understood from Table 2, as a result of a comparison of the medical fluid nozzles 1 of the embodiments described above with the nozzle C of Comparative example 1 and the nozzle 29 of Comparative example 2, even when the dropping angle “f” was 30°, which is an angle more nearly horizontal, the effect of suppressing fluid drops from remaining by the ridge 10 of the tip portion of the nozzle has been confirmed. It has also been confirmed that as compared with other nozzles, the medical fluid nozzle 1 of Embodiment 1 described above has a particular effect of suppressing fluid drops from remaining by the ridge 10 of the tip portion of the nozzle.

1. A medical fluid nozzle which is a cylindrical medical fluid nozzle to be attached to a spout of a medical fluid container inside of which a medical fluid is house, for causing the medical fluid to fall in drops,
being formed in a substantially planar shape at a tip surface thereof, and
being formed at a tip portion thereof with an annular ridge along a circumferential direction,
the ridge being formed so as to be continuous with the tip surface, and having an extended surface formed with an increased diameter so as to be flush with the tip surface or formed increased in diameter toward a base end side.

2. The medical fluid nozzle according to claim 1, wherein the ridge has an external shape that is increased in diameter so as to be flush with the tip surface, extends to a base end side, and is then reduced in diameter toward the base end side.

3. The medical fluid nozzle according to claim 1, wherein the ridge has an external shape that is increased in diameter so as to be flush with the tip surface and is then reduced in diameter toward a base end side.

4. The medical fluid nozzle according to claim 1, wherein the ridge has an external shape that is increased in diameter toward a base end side and is then reduced in diameter toward the base end side.

5. An eye drop container according to claim 1 comprising the medical fluid nozzle.

6. A method for preventing dripping from a medical fluid nozzle according to claim 1 for which the medical fluid nozzle is attached to a spout of a medical fluid container.

7. An eye drop container according to claim 2 comprising the medical fluid nozzle.

8. An eye drop container according to claim 3 comprising the medical fluid nozzle.

9. An eye drop container according to claim 4 comprising the medical fluid nozzle.

10. A method for preventing dripping from a medical fluid nozzle according to claim 2 for which the medical fluid nozzle is attached to a spout of a medical fluid container.

11. A method for preventing dripping from a medical fluid nozzle according to claim 3 for which the medical fluid nozzle is attached to a spout of a medical fluid container.

12. A method for preventing dripping from a medical fluid nozzle according to claim 4 for which the medical fluid nozzle is attached to a spout of a medical fluid container.
